(19) United States
(12) Patent Application Publication
(10) Pub. No.: US 2005/0049240 A1
(43) Pub. Date: Mar. 3, 2005

(54) Tetrahydroisochinolines, their production and the use thereof as analgesics

(75) Inventors: Nils Gribenow, Dormagen (DE); Bernd Kalthof, Wuppertal (DE); Heinrich Meier, Wuppertal (DE); Stephan-Nicholas Wirtz, Wuppertal (DE); Peter Spreyer, Radevormwald (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer HealthCare AG, Leverkusen (DE)

(21) Appl. No.: 10/490,193

(22) PCT Filed: Sep. 11, 2002
(86) PCT No.: PCT/EP02/10159

(30) Foreign Application Priority Data
Sep. 24, 2001 (DE) 101 46 867.9

(51) Int. Cl. A61K 31/55; A61K 31/496; A61K 31/4709; C07D 43/02; C07D 41/02

(52) U.S. Cl. 514/217.07; 514/253.05; 514/309; 540/596; 544/363; 546/141

(57) ABSTRACT

The invention relates to novel tetrahydroisoquinolines, to processes for their preparation and to their use for producing pharmaceuticals for the treatment and/or prophylaxis of diseases, in particular of states of pain.

The invention relates to novel tetrahydroisoquinolines, to processes for their preparation and to their use for producing pharmaceuticals for the treatment and/or prophylaxis of diseases, in particular of states of pain.

Neurotensin is a biologically active peptide having a length of 13 amino acids. In accordance with its dual function as neurotransmitter and neuromodulator, neurotensin is found both in the central nervous system and in peripheral tissues. The biological activity of neurotensin is mediated by neurotensin receptors on the cell surface of the target tissue. Currently, three receptors (NT-1, NT-2 and NT-3) are known which differ in their molecular structure and their pharmacological properties (Vincent et al., TiPS 1999, 20, 302-309).

The neurotensin receptor NT-2 is a G-protein-coupled receptor and is expressed predominantly in the brain (Vita et al., Eur. J. Pharmacol. 1998, 360, 265-272). The signal transduction is effected via inositol triphosphate-mediated calcium release.

While neurotensin exerts an agonistic action on the human NT-1 receptor, neurotensin has an antagonistic effect on the human NT-2 receptor.

The central administration of neurotensin to mice and rats in vivo exhibits a series of pharmacological effects, such as appetite reduction (Stanley et al., Peptides 1983, 4, 493-500), hypothermia and analgesia (Tyler et al., Brain Res. 1998, 792, 246-252).

While the appetite-suppressing and temperature-reducing properties of neurotensin are probably mediated via the NT-1 receptor or neurotensin receptors which are as yet unknown, it has been shown with the aid of antisense experiments that the analgesic action of neurotensin is with some certainty mediated by the NT-2 receptor (Dubuc et al., J. Neurosci. 1999, 19, 503-510). NT-2 antagonists should therefore be suitable for treating states of pain.

U.S. Pat. No. 5,874,443, WO 97/16428 and WO 00/50406 describe chemical libraries containing tetrahydroisoquinolines for finding therapeutically active compounds.

The present invention relates to compounds of the general formula (I) 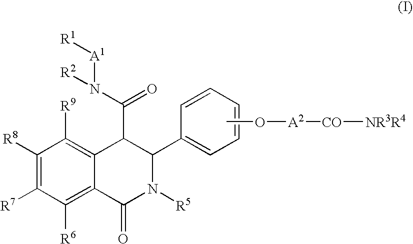
in which
R¹ is phenyl or 5- or 6-membered heteroaryl, where phenyl and heteroaryl may optionally be identically or differently substituted by radicals selected from the group of halogen, formyl, carbamoyl, cyano, hydroxyl, trifluoromethyl, trifluoromethoxy, nitro, (C₁-C₆)alkyl, (C₁-C₆)alkoxy and (C₁-C₆)alkylthio,
A¹ is a bond or (C₁-C₆)alkanediyl,
R² is hydrogen or (C₁-C₆)alkyl,
R³ and R⁴ are the same or different and are each hydrogen, (C₁-C₈)alkyl or (C₃-C₈)cycloalkyl,
or
R³ and R⁴ together with the adjacent nitrogen atom are a radical selected from the group of pyrrolidyl, piperidyl, azepinyl and piperazinyl, where the radicals may optionally be substituted by methyl or ethyl,
A² is (C₁-C₆)alkanediyl,
R⁵ is (C₁-C₈)alkyl which may optionally be substituted by a radical selected from the group of trifluoromethyl, halogen, saturated or partly unsaturated (C₃-C₈)cycloalkyl and (C₆-C₁₀)aryl, where aryl may itself optionally be substituted identically or differently by radicals selected from the group of halogen, formyl, carbamoyl, cyano, hydroxyl, trifluoromethyl, trifluoromethoxy, nitro, (C₁-C₆)alkyl, (C₁-C₆)alkoxy and (C₁-C₆)alkylthio,
and
R⁶, R⁷, R⁸ and R⁹ are the same or different and are each hydrogen, halogen, formyl, carbamoyl, cyano, hydroxyl, trifluoromethyl, trifluoromethoxy, nitro, (C₁-C₆)alkyl, (C₁-C₆)alkoxy or (C₁-C₆)alkylthio,
and their salts, hydrates and/or solvates.

The compounds according to the invention may exist in stereoisomeric forms which either behave like image and mirror image (enantiomers) or do not behave like image and mirror image (diastereomers). The invention relates both to the enantiomers or diastereomers and to their particular mixtures. These mixtures of the enantiomers and diastereomers can be separated in a known manner into the stereoisomerically uniform constituents.

The compounds according to the invention may also be present in the form of their salts, hydrates and/or solvates.

In the context of the invention, preferred salts are physiologically acceptable salts of the compounds according to the invention.

Physiologically acceptable salts of the compounds according to the invention may be acid addition salts of the compounds with mineral acids, carboxylic acids or sulfonic acids. Particular preference is given, for example, to salts with hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, citric acid, fumaric acid, maleic acid or benzoic acid.

However, salts may also include salts with customary bases, for example alkali metal salts (e.g. sodium or potassium salts), alkaline earth metal salts (e.g. calcium or magnesium salts) or ammonium salts, derived from ammonia or organic amines, for example diethylamine, triethylamine, ethyldiisopropylamine, procaine, dibenzylamine, N-methylmorpholine, dihydroabietylamine, 1-ephenamine or methylpiperidine.

Hydrates of the compounds according to the invention are stoichiometric compositions of the compounds or their salts with water.

Solvates of the compounds according to the invention are stoichiometric compositions of the compounds or their salts with water.

In the context of the present invention, the substituents are generally defined as follows:

(C₁-C₆)Alkanediyl is a straight-chain or branched alkanediyl radical having from 1 to 6 carbon atoms. Preference is given to a straight-chain or branched alkanediyl radical having from 1 to 4 carbon atoms. Preferred examples include methylene, ethylene, propylene, propane-1,2-diyl, propane-2,2-diyl, butane-1,3-diyl, butane-2,4-diyl, pentane-2,4-diyl, 2-methylpentane-2,4-diyl.

When a methylene group of the alkanediyl radical is optionally replaced by an oxygen or sulfur atom, preferred examples include: —O—, —S—, —O—CH₂—, —S—CH₂—, —CH₂—O—, —CH₂—S—, —CH₂—O—CH₂—, —O—CH₂—CH₂—, 1-oxapropane-1,2-diyl, 2-oxabutane-2,4-diyl, 3-thiabutane-2,4-diyl.

(C₁-C₆)Alkoxy is a straight-chain or branched alkoxy radical having from 1 to 6 carbon atoms. Preference is given to a straight-chain or branched alkoxy radical having from 1 to 4, particular preference to one having from 1 to 3, carbon atoms. Preferred examples include: methoxy, ethoxy, n-propoxy, isopropoxy, tert-butoxy, n-pentoxy and n-hexoxy.

(C₁-C₈)- and (C₁-C₆)alkyl are a straight-chain or branched alkyl radical having from 1 to 8 and from 1 to 6 carbon atoms respectively. Preference is given to a straight-chain or branched alkyl radical having from 1 to 4, particular preference to one having from 1 to 3, carbon atoms. Preferred examples include: methyl, ethyl, n-propyl, isopropyl, tert-butyl, n-pentyl, n-hexyl, n-heptyl and n-octyl.

(C₁-C₆)Alkylthio is a straight-chain or branched alkylthio radical having from 1 to 6 carbon atoms. Preference is given to a straight-chain or branched alkylthio radical having from 1 to 4, particular preference to one having from 1 to 3, carbon atoms. Preferred examples include: methylthio, ethylthio, n-propylthio, isopropylthio, tert-butylthio, n-pentylthio and n-hexylthio.

(C₃-C₈)Cycloalkyl is cyclopropyl, cyclopentyl, cyclobutyl, cyclohexyl, cycloheptyl or cyclooctyl. Preferred examples include: cyclopropyl, cyclopentyl and cyclohexyl.

Partly unsaturated cycloalkyl radicals are nonaromatic cycloalkyl radicals which contain one or more multiple bonds, preferably double bonds. Preferred examples include: cyclopentenyl, cyclohexenyl and cycloheptenyl.

Halogen is fluorine, chlorine, bromine and iodine. Preference is given to fluorine, chlorine and bromine. Particular preference is given to fluorine and chlorine.

5- to 6-membered heteroaryl is an aromatic radical having from 5 to 6 ring atoms and up to 4 heteroatoms from the group of S, O and/or N. The heteroaryl radical may be bonded via a carbon atom or heteroatom. Preferred examples include: thienyl, furyl, pyrrolyl, thiazolyl, oxazolyl, imidazolyl, tetrazolyl, pyridyl, pyrimidinyl, and pyridazinyl.

When radicals in the process according to the invention are optionally substituted, the radicals, unless specified otherwise, may be mono- or polysubstituted identically or differently. Preference is given to substitution by up to three identical or different substituents.

Preference is given to compounds of the general formula (I) in which the substituents in positions 3 and 4 on the 1,2,3,4-tetrahydroisoquinoline are trans relative to each other. The relative trans-configuration of the compounds according to the invention may be illustrated by way of example by the general formula (Ia):

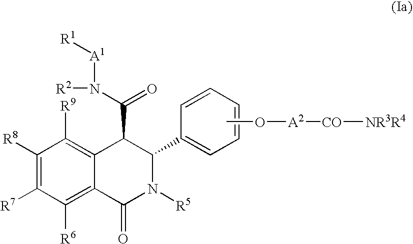

Preference is also given to compounds of the general formula (Ib) 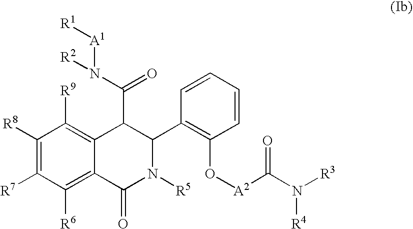
in which R¹, A¹, R², R³, R⁴, A², R⁵, R⁶, R⁷, R⁸ and R⁹ are each as defined above.

Preference is likewise given to compounds of the general formula (I) in which R¹ is 3-pyridyl and A¹, R², R³, R⁴, A², R⁵, R⁶, R⁷, R⁸ and R⁹ are each as defined above.

Preference is likewise given to compounds of the general formula (I) in which A¹ is methylene and R¹, R², R³, R⁴, A², R⁵, R⁶, R⁷, R⁸ and R⁹ are each as defined above.

Preference is likewise given to compounds of the general formula (I) in which R² is hydrogen and R¹, A¹, R³, R⁴, A², R⁵, R⁶, R⁷, R⁸ and R⁹ are each as defined above.

Preference is likewise given to compounds of the general formula (I) in which R³ and R⁴ are each hydrogen and R¹, A¹, R², A², R⁵, R⁶, R⁷, R⁸ and R⁹ are each as defined above.

Preference is likewise given to compounds of the general formula (I) in which A² is methylene and R¹, A¹, R², R³, R⁴, R⁵, R⁶, R⁷, R⁸ and R⁹ are each as defined above.

Preference is likewise given to compounds of the general formula (I) in which
R⁵ is (C₁-C₃)alkyl which is substituted by a radical selected from the group of trifluoromethyl, unsaturated or monounsaturated (C₅-C₇)cycloalkyl and optionally methyl-, halogen- or methoxy-substituted phenyl, or
is (C₄-C₆)alkyl which is optionally substituted by trifluoromethyl,
   and R¹, A¹, R², R³, R⁴, A², R⁶, R⁷, R⁸ and R⁹ are each as defined above.

Preference is likewise given to compounds of the general formula (I) in which R⁶, R⁷, R⁸ and R⁹ are the same or different and are each hydrogen, methyl, fluorine, chlorine or methoxy, more preferably hydrogen.

Particular preference is given to combinations of two or more of the abovementioned areas of preference.

Very particular preference is given to compounds of the general formula (I) in which
R¹ is 3-pyridyl which is optionally identically or differently substituted by chlorine, fluorine or methyl,
A¹ is methylene,
R² is hydrogen,
R³ and R⁴ are each hydrogen,
A² is methylene,
R⁵ is (C₁-C₃)alkyl which is substituted by a radical selected from the group of trifluoromethyl, unsaturated or monounsaturated (C₅-C₇)cycloalkyl and optionally methyl-, halogen- or methoxy-substituted phenyl, or
is (C₄-C₆)alkyl which is optionally substituted by trifluoromethyl, and
R⁶, R⁷, R⁸ and R⁹ are each hydrogen,
and their salts, hydrates and/or solvates.

The invention further relates to a process for preparing compounds of the formula (I), characterized in that compounds of the general formula (II)

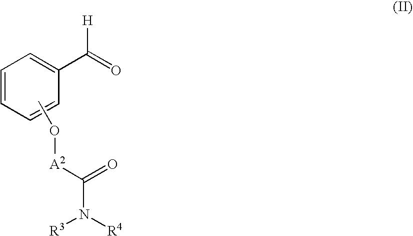

in which

A², R³ and R⁴ are each as defined above

are initially reacted, in the presence of water-withdrawing agents, for example sodium sulfate or orthoformic esters, preferably trimethyl orthoformate, with amines of the general formula (III)
R⁵—NH₂ (III)

in which

R⁵ is as defined above

to give compounds of the general formula (IV)

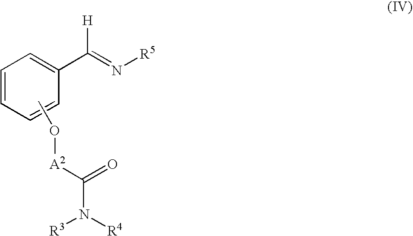

in which

A², R³, R⁴ and R⁵ are each as defined above,

then, in situ or with preceding isolation as an intermediate, (IV) is converted, optionally with the addition of an auxiliary base, using a homophthalic anhydride of the general formula (V)

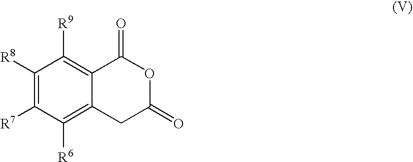

in which

R⁶, R⁷, R⁸ and R⁹ are each as defined above

to give compounds of the general formula (VI)

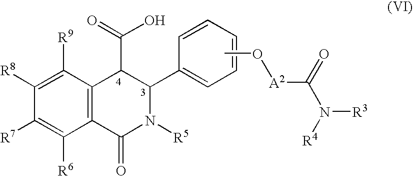

in which

A², R³, R⁴, R⁵, R⁶, R⁷, R⁸ and R⁹ are each as defined above,

optionally epimerized by heating in the presence of an acid to the relative transconfiguration of positions 3 and 4 in the tetrahydroisoquinoline ring, as illustrated in the general formula (Ia), and finally reacted with activation of the carboxylic acid group in (VI) with compounds of the general formula (VII)
R¹—A¹—NH—R² (VII)

in which

A¹, R¹ and R² are each as defined above.

The process according to the invention is generally carried out at atmospheric pressure. However, it is also possible to carry out the process at elevated pressure or at reduced pressure (for example within a range from 0.5 to 5 bar).

Suitable solvents for the process are customary organic solvents which do not change under the reaction conditions. These include ethers such as diethyl ether, dioxane, tetrahydrofuran, glycol dimethyl ether, or hydrocarbons such as benzene, toluene, xylene, hexane, cyclohexane or crude oil fractions, or halohydrocarbons such as dichloromethane, trichloromethane, tetrachloromethane, dichloroethylene, trichloroethylene or chlorobenzene, or ethyl acetate, pyridine, dimethyl sulfoxide, dimethylformamide, N,N′-dimethylpropyleneurea (DMPU), N-methylpyrrolidone (NMP), acetonitrile, acetone or nitromethane. It is equally possible to use mixtures of the solvents mentioned.

Preferred solvents for the process steps (II)+(III)→(IV) and (IV)+(V)→(VI) are dichloromethane, 1,2-dichloroethane, toluene or mixtures of these solvents. For the process step (VI)+(VII)→(I), preference is given to dichloromethane or dimethylformamide.

The process steps (II)+(III)→(IV) and (IV)+(V)→(VI) according to the invention are generally carried out within a temperature range of from 0° C. to +60° C., preferably from +20° C. to +40° C. The process step (VI) +(VII)→(I) is generally carried out within a temperature range of from −20° C. to +40° C., preferably from 0° C. to +25° C.

Useful auxiliaries for the amide formation in the process step (VI)+(VII)→(I) are preferably customary condensing agents such as carbodiimides, e.g. N,N′-diethyl-, N,N′-dipropyl-, N,N′-diisopropyl-, N,N′-dicyclohexylcarbodiimide (DCC), N-(3-dimethylaminoisopropyl)-N′-ethylcarbodiimide hydrochloride (EDC.HCl), or carbonyl compounds such as carbonyldiimidazole, or 1,2-oxazolium compounds such as 2-ethyl-5-phenyl-1,2-oxazolium 3-sulfate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxytris(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), optionally in combination with further auxiliaries such as 1-hydroxybenzotriazole or N-hydroxysuccinimide, and also, as bases, alkali metal carbonates, e.g. sodium or potassium carbonate or hydrogencarbonate, or organic bases such as trialkylamines, e.g. triethylamine, N-methylmorpholine, N-methylpiperidine or diisopropylethylamine. Particular preference is given to the combination of EDC, 1-hydroxybenzotriazole and N-methylmorpholine or triethylamine.

Suitable auxiliary bases for the reaction (IV)+(V)→(VI) are the customary organic bases such as trialkylamines, e.g. triethylamine, N-methylmorpholine, N-methylpiperidine or diisopropylethylamine. Preference is given to triethylamine.

Suitable acids for epimerizing to the relative trans-configuration of positions 3 and 4 in the compounds of the general formula (VI) are generally trifluoroacetic acid, sulfuric acid, hydrogen chloride, hydrogen bromide and acetic acid or mixtures thereof, optionally with the addition of water. Preference is given to acetic acid. The epimerization is generally carried out within a temperature range of from +20° C. to +150° C., preferably from +80° C. to +120° C.

The compounds of the general formula (II) are known or may be prepared, for example, by

[A] reacting compounds of the general formula (VIII)

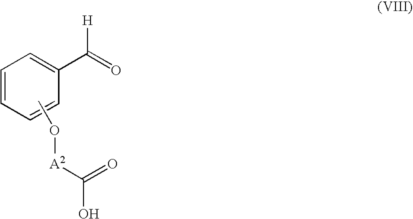

in which

A² is as defined above,

with activation of the carboxylic acid group, with amines of the general formula (IX)
R³—NH—R⁴ (IX)

in which

R³ and R⁴ are each as defined above,

or

[B] reacting hydroxybenzaldehydes of the general formula (X)

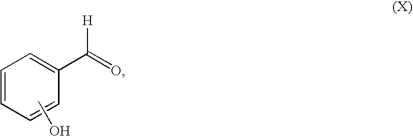

in the presence of a base, with compounds of the general formula (XI)

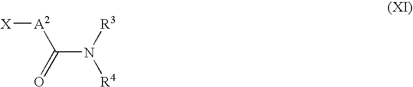

in which

A², R³ and R⁴ are each as defined above,

and

X is a suitable leaving group, for example halogen, mesylate or tosylate, preferably bromine or iodine.

The amide formation (VIII)+(IX)→(II) is effected in a similar manner to the above-described process step (VI)+(VII)→(I), preferably in dichloromethane or dimethylformamide as a solvent, using the combination of EDC, 1-hydroxybenzotriazole and N-methylmorpholine or triethylamine.

The reaction (X)+(XI)→(II) is preferably carried out in dimethylformamide as a solvent, generally within a temperature range of from 0° C. to +120° C., preferably from +60° C. to +80° C.

Suitable bases for the reaction (X)+(XI)→(II) are the customary inorganic or organic bases. These preferably include alkali metal hydroxides, for example lithium, sodium or potassium hydroxide, alkali metal or alkaline earth metal carbonates such as sodium, potassium or calcium carbonate, alkali metal alkoxides such as sodium or potassium methoxide, sodium or potassium ethoxide, or potassium tert-butoxide, alkali metal hydrides such as sodium hydride, amides such as sodium amide, lithium bis(trimethylsilyl)amide or lithium diisopropylamide, or organic amines such as pyridine, triethylamine, ethyldiisopropylamine, N-methylmorpholine or N-methylpiperidine. Preference is given to potassium tert-butoxide.

The compounds of the general formulae (III), (V), (VII), (VIII), (IX), (X) and (XI) are commercially available, known or can be prepared by known processes.

The process according to the invention can be illustrated by the following synthesis scheme:

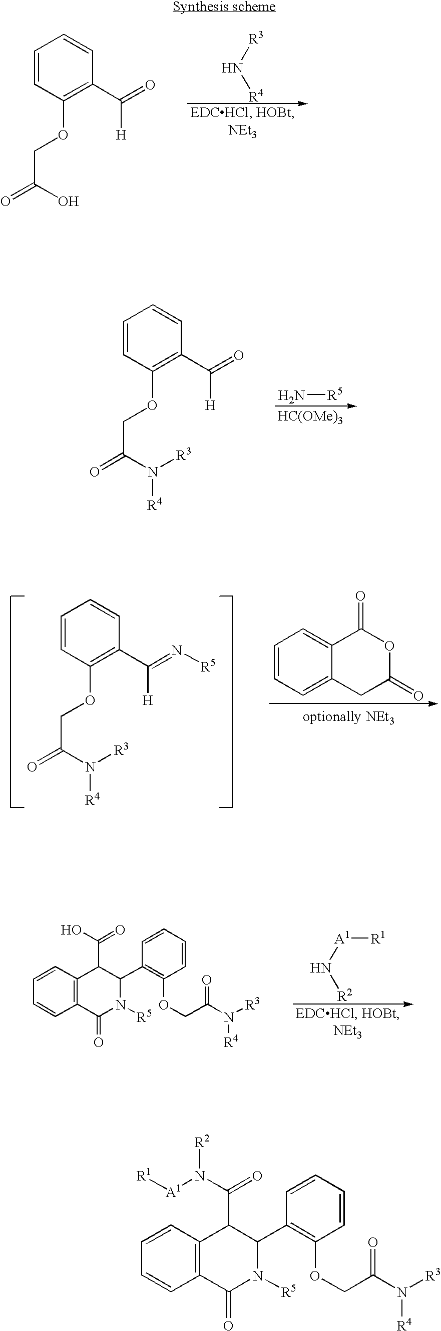

The compounds of the general formula (I) according to the invention are suitable for use as pharmaceuticals for the treatment and/or prophylaxis of diseases in humans and/or animals.

The compounds according to the invention exhibit an unforeseeable, valuable spectrum of pharmacological activity.

They have an antagonistic action on the NT2 receptor.

As a consequence of their pharmacological properties, the compounds according to the invention may be used alone or in combination with other pharmaceuticals for treatment and/or prevention, especially of states of pain.

The in vitro action of the compounds according to the invention may be shown by the following assay:

NT2 Antagonism Assay

The activation of the human neurotensin-2 (NT-2) receptor by an agonist, for example SR48692, 2-{[1-(7-chloroquinolin-4-yl)-5-(2,6-dimethoxyphenyl)pyrazol-3-yl]carbonylamino}tricyclo[3.3.3.1 ^{3,7}]decane-2-carboxylic acid, leads, via stimulation of phospholipase C, to the release of calcium ions from intracellular stores. Antagonists block the activation of the receptor by the agonist and therefore also the agonist-dependent stimulation of phospholipase C and the intracellular calcium release initiated thereby.

A functional in vitro assay may be carried out with stable cell lines, for example CHO or HEK 293, which express the human NT-2 receptor recombinantly. The activity of the receptor is determined via measurement of the intracellular calcium release coupled thereto (in microtiter plates having 96, 384 and 1536 wells/plate). The concentration-dependent action of the NT-2 ligands tested on the receptor activity may be reported as IC₅₀.

The enantiomer B of example 1 has, for example, an IC₅₀ value of less than 0.2 μM.

The suitability of the compounds according to the invention for treating states of pain can be demonstrated in suitable animal models.

The compounds of the general formula (I) according to the invention are suitable for use as pharmaceuticals for humans and animals.

The present invention also includes pharmaceutical preparations which comprise, in addition to inert, nontoxic, pharmaceutically suitable auxiliary and carrier substances, one or more compounds of the general formula (I), or which consist of one or more compounds of the formula (I), and also processes for producing these preparations.

The compounds of the formula (I) should be present in these preparations in a concentration of from 0.1 to 99.5% by weight, preferably from 0.5 to 95% by weight, of the overall mixture.

In addition to the compounds of the formula (I), the pharmaceutical preparations may also comprise other active pharmaceutical ingredients.

The above-detailed pharmaceutical preparations may be prepared in the customary manner by known methods, for example with the auxiliary or carrier substance or substances.

The novel active ingredients may be converted in a known manner to the customary formulations, such as uncoated or coated tablets, pills, granules, aerosols, syrups, emulsions, suspensions and solutions, using inert, nontoxic, pharmaceutically suitable carrier substances or solvents. The therapeutically active compound should in each case be present here in a concentration of from about 0.5 to 90% by weight of the overall mixture, i.e. in amounts which are sufficient to achieve the dosage range specified.

The formulations are prepared, for example, by extending the active ingredients with solvents and/or carrier substances, optionally using emulsifiers and/or dispersants, in which case, for example in the case of the use of water as a diluent, organic solvents may optionally be used as auxiliary solvents.

The administration is effected in a customary manner, preferably orally, transdermally or parenterally, in particular perlingually or intravenously. However, administration may also be by inhalation via mouth or nose, for example with the aid of a spray, or topically via the skin.

In general, it has been found to be advantageous to administer amounts of from about 0.001 to 10 mg/kg, or in the case of oral use, preferably from about 0.005 to 3 mg/kg, of bodyweight to achieve effective results.

In spite of this, it may in some cases be necessary to deviate from the amounts specified, depending on bodyweight or the type of the administration route, on the individual behavior toward the medicament, on the type of its formulation and on the time or interval at which the administration is effected. For instance, it may be sufficient in some cases to manage with less than the aforementioned minimum amount, while in other cases the upper limit mentioned has to be exceeded. In the case of the administration of relatively large amounts, it may be advisable to divide them into a plurality of individual doses over the day.

Abbreviations:
DCI direct chemical ionization (in MS)
DMF N,N-dimethylformamide
DMSO dimethyl sulfoxide
ESI electrospray ionization (in MS)
HPLC high-pressure, high-performance liquid chromatography
LC-MS liquid chromatography-coupled mass spectroscopy
MS mass spectroscopy
NMR nuclear magnetic resonance spectroscopy
RP reverse phase
RT room temperature
Rₜ retention time (in HPLC)
   Starting Compounds and Intermediates:

### EXAMPLE I

### 2-(2-Formylphenoxy)acetamide

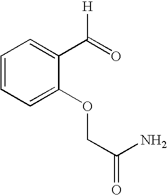

10.9 g (97.2 mmol) of potassium tert-butoxide are added to a solution of 13.1 g (107 mmol) of salicylaldehyde in DMF (400 ml), then the mixture is stirred at 60° C. for 60 min. 13.7 g of 2-bromoacetamide (98 percent, corresponds to 97.2 mmol) in DMF (100 ml) are then added dropwise and the mixture is left to stir at 80° C. overnight. After cooling, the solvent is removed under reduced pressure and the residue partitioned between ethyl acetate and water. The phases are separated and the aqueous phase is extracted three times with ethyl acetate. The combined organic phases are washed with 1 N hydrochloric acid, water, saturated sodium hydrogencarbonate solution and saturated sodium chloride solution, and dried over magnesium sulfate. The solvent is removed under reduced pressure and the residue recrystallized from ethyl acetate. This results in 13.4 g (77%) of the target compound as crystals having a melting point of 127° C.

MS (DCI/NH₃): 197 (M+NH₄)⁺ HPLC: Rₜ=3.13 min ¹H NMR (400 MHz, CDCl₃): δ=4.59 (s, 2H), 5.79 (s, br, 1H), 6.95 (d, 1H), 7.20 (t, 1H), 7.56 (s, br, 1H), 7.61 (td, 1H), 7.79 (dd, 1H), 10.18 (s, 1H).

### EXAMPLE II

### 2-[2-(4-Methyl-1-piperazinyl)-2-oxoethoxy]benzaldehyde

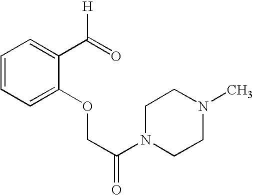

5.00 g (27.8 mmol) of 2-(2-formylphenoxy)acetic acid, 2.78 g (27.8 mmol) of 1-methylpiperazine and 3.75 g (27.8 mmol) of 1-hydroxy-1H-benzotriazole are dissolved in 20 ml of dichloromethane and, after addition of 5.62 g (55.5 mmol) of triethylamine and 6.38 g (33.3 mmol) of N-(3-dimethylaminopropyl)-N′-ethylcarbodiimide hydrochloride, stirred at RT overnight. For workup, the reaction mixture is diluted with dichloromethane, washed twice with water, dried and concentrated. 8.12 g of the target compound remain as a brown solid in an HPLC purity of 89% (yield virtually quantitative).

MS (DCI/NH₃): 263 (M+H)⁺ HPLC: Rₜ=2.93 min ¹H NMR (300 MHz, DMSO-d₆): δ=2.1-2.4 (m, 6H), 2.28 (s, 3H), 3.45 (t, 4H), 5.07 (s, 2H), 7.08 (t, 1H), 7.14 (d, 1H), 7.62 (ddd, 1H), 7.70 (dd, 1H), 10.45 (s, 1H).

The following were obtained in a similar manner:

### EXAMPLE III

### 2-[2-Oxo-2-(1-piperidinyl)ethoxy]benzaldehyde

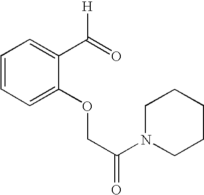

MS (ESIpos): 248 (M+H)⁺ HPLC: Rₜ=3.87 min ¹H NMR (200 MHz, DMSO-d₆): δ=1.3-1.7 (m, 6H), 3.3-3.5 (m, 4H), 5.05 (s, 2H), 7.0-7.2 (m, 2H), 7.55-7.75 (m, 2H), 10.46 (s, 1H).

### EXAMPLE IV

### N-Isopropyl-2-(2-formylphenoxy)acetamide

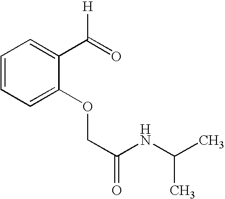

MS (DCI/NH₃): 239 (M+NH₄)⁺ HPLC: Rₜ=3.63 min ¹H NMR (300 MHz, DMSO-d₆): δ=1.10 (d, 6H), 3.96 (m, 1H), 4.63 (s, 2H), 7.08-7.18 (m, 2H), 7.65 (td, 1H), 7.75 (dd, 1H), 8.00 (d, br, 1H), 10.41 (s, 1H).

### EXAMPLE V

### 3,4-trans-3-[2-(Carbamoylmethoxy)phenyl]-2-[2-(1-cyclohexen-1-yl)ethyl]-1-oxo-1,2,3,4-tetrahydro-4-isoquinolinecarboxylic acid

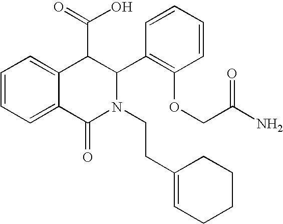

4.99g (39.9mmol) of 2-(cyclohex-1-enyl)ethylamine are dissolved in 40 ml of dichloromethane and admixed at RT with 6.50 g (61.2 mmol) of trimethyl orthoformate and 6.93 g (38.7 mmol) of 2-(2-formylphenoxy)acetamide. The mixture is stirred overnight, then 5.33 g (32.9 mmol) of homophthalic anhydride are added.

The reaction mixture heats up slightly and, after a short time, a colorless solid precipitates out. The mixture is stirred for a further 16 h and filtered, and the residue is washed with a little dichloromethane. The dried crude product contains a cis/trans isomer mixture of the structural formula specified and is epimerized by boiling for 6 hours in glacial acetic acid to give substantially uniform trans-isomer. After cooling, the acetic acid is removed under reduced pressure, and the brown residue is dissolved in dichloromethane and washed with water and also saturated sodium chloride solution. The product is adsorbed on silica gel and further purified by flash chromatography (dichloromethane/methanol gradient 95:5-5:1), then recrystallized from water/isopropanol. 4.28 g (29%) of the target compound remain in 96 percent purity by HPLC.

MS (ESIpos): 449 (M+H)⁺ HPLC: Rₜ=4.19 min ¹H NMR (300 MHz, DMSO-d₆): δ=1.4-1.6 (m, 4H), 1.8-2.0 (s, br, 4H), 2.16 (m, 2H), 2.76 (m, 1H), 4.03 (m, 1H), 4.59 (AB system, 2H), 5.38 (s, br, 1H), 5.68 (s, 1H), 6.56 (d, 1H), 6.76 (t, 1H), 6.97 (d, 1H), 7.1-7.25 (m, 2H), 7.3-7.45 (m, 2H), 7.48 (s, br, 1H), 7.61 (s, br, 1H), 7.88-7.98 (m, 1H), 13.05 (s, br, 1H).

The racemic product can be separated into the enantiomers by preparative high-pressure liquid chromatography on chiral carrier material (column: Daicel Chiralpak AS 10 μgm, 250×20 mm, eluent: 90% acetonitrile with 0.2% trifluoroacetic acid/10% ethanol, flow rate: 10 ml/min, injection volume: 1000 μl, temperature: 40° C.).

Enantiomer A: Rₜ=5.14 min; [α]_{D}^{20.1} (c=0.55; CHCl₃)=−23.4 Enantiomer B: Rₜ=7.01 min; [α]_{D}^{20.0} (c=0.55; CHCl₃)=+27.5 (Column: Daicel Chiralpak AS 10 μm 250×4.6 mm, eluent: acetonitrile with 0.2% trifluoroacetic acid, flow rate: 1 ml/min, injection volume: 3 μl, temperature: 40° C.).

### EXAMPLE VI

### 3,4-trans-3-[2-(Carbamoylmethoxy)phenyl]-2-(2-phenylethyl)-1-oxo-1,2,3,4-tetra-hydro-4-isoquinolinecarboxylic acid

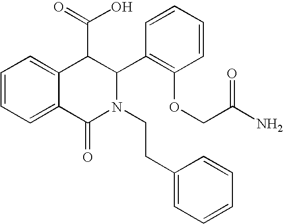

First Reaction Step:

A solution of 0.75 g (6.17 mmol) of 2-phenylethylamine and 1.11 g (6.17 mmol) of 2-(2-formylphenoxy)acetamide is stirred in 10 ml trimethyl orthoformate at RT overnight. The mixture is taken up three times in twice the volume of toluene and in each case concentrated under reduced pressure.

Second Reaction Step:

The residue is dissolved in 30 ml of dichloroethane and admixed with 1.25 g (12.3 mmol) of triethylamine and 1.00 g (6.17 mmol) of homophthalic anhydride. The reaction mixture is left to stir overnight, diluted with dichloromethane and extracted with water. The organic phase is washed with saturated sodium chloride solution, dried over sodium sulfate and concentrated under reduced pressure. The residue is chromatographed on an RP column with acetonitrile/water (HPLC, 30:70-95:5). The crude product obtained in this way as an epimer mixture is epimerized by boiling in glacial acetic acid (10 ml) overnight. The solution is added to 100 ml of water and the mixture extracted with ethyl acetate. The organic phase is dried over sodium sulfate and concentrated under reduced pressure. To remove acid residues, the mixture is repeatedly taken up in toluene and concentrated under reduced pressure. The yellow residue is triturated in acetone, filtered off and dried under reduced pressure. 527 mg (19%) of the target compound are obtained in this way as a colorless solid. Concentration by evaporation of the mother liquor provides a second fraction of lower purity as a yellowish solid: 910 mg, 65% percent by HPLC.

¹H NMR (400 MHz, CDCl₃): δ=2.65-3.05 (m, 4H), 4.05-4.25 (m, 1H), 4.34 (s, 1H), 4.61 (AB system, 2H), 5.83 (s, 1H), 6.57 (d, 1H), 6.75 (t, 1H), 6.97 (d, 1H), 7.1-7.3 (m, 6H), 7.3-7.58 (m, 2H), 7.55 (s, br, 1H), 7.64 (s, br, 1H), 7.88-8.0 (m, 1H), 13.15 (s, br, 1H).

In a similar manner, the following compounds were prepared:

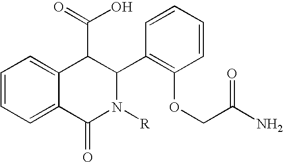

### EXAMPLE VII

### 3,4-trans-3-[2-(Carbamoylmethoxy)phenyl]-2-[2-(3,4-dimethoxyphenyl)ethyl]-1-oxo-1,2,3,4-tetrahydro-4-isoquinolinecarboxylic acid

Starting from 2-(3,4-dimethoxyphenyl)ethylamine [R=2-(3,4-dimethoxyphenyl)ethyl]:

LC-MS (method B): Rₜ=2.30 min MS (ESIpos): m/z=505 (M+H)⁺.

### EXAMPLE VIII

### 3,4-trans-3-[2-(Carbamoylmethoxy)phenyl]-2-[2-(4-methylphenyl)ethyl]-1 -oxo-1,2,3,4-tetrahydro-4-isoquinolinecarboxylic acid

Starting from 2-(4-methylphenyl)ethylamine [R=2-(4-methylphenyl)ethyl]:

LC-MS (method B): Rₜ=2.59 min MS (ESIpos): m/z=459 (M+H)⁺.

### EXAMPLE IX

### 3,4-trans-3-[2-(Carbamoylmethoxy)phenyl]-2-[2-(2-methoxyphenyl)ethyl]-1 -oxo-1,2,3,4-tetrahydro-4-isoquinolinecarboxylic acid

Starting from 2-(2-methoxyphenyl)ethylamine [R=2-(2-methoxyphenyl)ethyl]:

LC-MS (method B): Rₜ=2.50 min MS (ESIpos): m/z=475 (M+H)⁺.

### EXAMPLE X

### 3,4-trans-3-[2-(Carbamoylmethoxy)phenyl]-1-oxo-1,2,3,4-tetrahydro-2-(3,3,3-trifluoropropyl)-4-isoquinolinecarboxylic acid

Starting from 3,3,3-trifluoropropylamine [R=3,3,3-trifluoropropyl]:

LC-MS (method B): Rₜ=2.37 min MS (ESIpos): m/z=437 (M+H)⁺.

### EXAMPLE XI

### 3,4-trans-2-n-Butyl-3-[2-(carbamoylmethoxy)phenyl]-1-oxo-1,2,3,4-tetrahydro-4-isoquinolinecarboxylic acid

Starting from n-butylamine with the addition of toluene to the trimethyl orthoformate in the first reaction step [R=n-butyl]:

LC-MS (method A): Rₜ=3.33 min MS (ESIpos): m/z=397 (M+H)⁺.

### EXAMPLE XII

### 3,4-trans-3-[2-(Carbamoylmethoxy)phenyl]-2-n-hexyl-1-oxo-1,2,3,4-tetrahydro-4-isoquinolinecarboxylic acid

Starting from n-hexylamine with the addition of toluene to the trimethyl orthoformate in the first reaction step [R=n-hexyl]:

LC-MS (method A): Rₜ=3.76 min MS (ESIpos): m/z=425 (M+H)⁺.

In a similar manner, but in dichloromethane as the solvent both for the first and for the second reaction step, the following were obtained:

### EXAMPLE XIII

### 3,4-trans-2-[2-(1-Cyclohexen-1-yl)ethyl]-3-{2-[2-(4-methyl-1-piperazinyl)-2-oxo-ethoxy]phenyl}-1-oxo-1,2,3,4-tetrahydro-4-isoquinolinecarboxylic acid

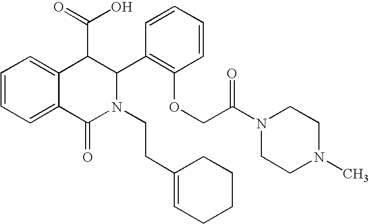

MS (ESIpos): 532 (M+H)⁺ HPLC: Rₜ=4.11 min

### EXAMPLE XIV

### 3,4-trans-2-[2-(1-Cyclohexen-1-yl)ethyl]-3-{2-[2-(isopropylamino)-2-oxoethoxy]-phenyl}-1-oxo-1,2,3,4-tetrahydro-4-isoquinolinecarboxylic acid

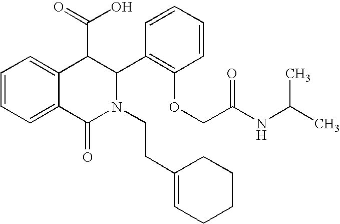

MS (ESIpos): 491 (M+H)⁺ HPLC: Rₜ=4.58 min

In a similar manner to the two aforementioned examples, but in toluene as the solvent for the first reaction step, the following was prepared:

### EXAMPLE XV

### 3,4-trans-2-[2-(1-Cyclohexen-1-yl)ethyl]-1-oxo-3-{2-[2-oxo-2-(1-piperidinyl)-ethoxy]phenyl}-1,2,3,4-tetrahydro-4-isoquinolinecarboxylic acid

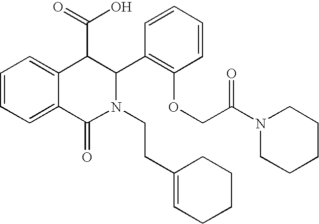

MS (ESIpos): 517 (M+H)⁺ ¹H NMR (MHz, DMSO-d₆): δ=1.3-1.7 (m, 10H), 1.8-2.0 (m, 4H), 2.05-2.35 (m, 2H), 2.65-2.85 (m, 1H), 3.35-3.48 (br, 1H), 3.98 (m, 4H), 4.59 (s, br, 1H), 5.38 (s, br, 1H), 5.69 (s, br, 1H), 6.5-6.6 (m, 1H), 6.71 (t, 1H), 6.94 (d, 1H), 7.0-7.45 (m, 6H), 7.85-7.95 (m, 1H).

### INVENTIVE EXAMPLE

### Example 1

### 3,4-trans-3-[2-(2-Carbamoylmethoxy)phenyl]-2-[2-(1-cyclohexen-1-yl)ethyl]-1-oxo-N-(3-pyridinylmethyl)-1,2,3,4-tetrahydro-4-isoquinolinecarboxamide

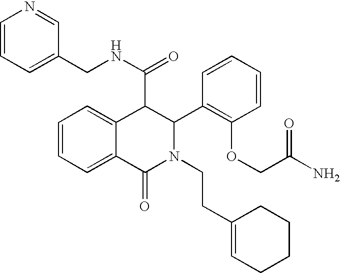

297 mg (2.74 mmol) of 3-picolylamine, 542 mg (2.83 mmol) of N-(3-dimethylaminopropyl)-N′-ethylcarbodiimide hydrochloride, 390 mg (2.89 mmol) of 1-hydroxy-1H-benzotriazole and 504 mg (4.99 mmol) of triethylamine are added to an ice-cold solution of 1.12 g (2.49 mmol) of 3-[2-(2-carbamoylmethoxy)phenyl]-2-[2-(1-cyclohexen-1-yl)ethyl]-1-oxo-1,2,3,4-tetrahydro-4-isoquinolinecarboxylic acid in anhydrous dichloromethane under argon. The reaction mixture is left to stir at RT overnight. The mixture is then diluted with dichloromethane, washed with water and saturated sodium chloride solution and dried over sodium sulfate. The mixture is concentrated under reduced pressure and the remaining residue is purified by chromatography (HPLC, acetonitrile/water 30:70-95:5). In this way, 1.04 g (78%) of the target compound are obtained as a white solid.

MS (DCI/NH₃): 539 (M+H)⁺ HPLC: Rₜ=4.16 min ¹H NMR (400 MHz, CDCl₃): δ=1.50-1.59 (m, 4H); 1.93 (s, 4H); 2.12 (m, 2H); 2.70-2.77 (m, 1H); 4.04 (s, 1H); 4.17-4.24 (m, 1H); 4.28-4.33 (dd, 1H); 4.43-4.48 (dd, 1H); 4.51-4.66 (AB system, 2H); 5.41 (s, 1H); 5.83 (s, 1H); 5.89-5.92 (t, 1H); 5.98 (s, 1H); 6.74-6.86 (m, 3H); 7.07 (d, 1H); 7.10-7.24 (m, 2H); 7.43 (m, 3H); 8.20-8.22 (m, 1H); 8.37 (m, 1H); 8.45 (m, 1H); 8.56 (s, 1H).

The racemic product can be separated into the enantiomers by preparative high-pressure liquid chromatography on chiral carrier material (column: Daicel Chiralpak AS 10 μm, 250×20 mm, eluent: 50% isohexane/50% isopropanol, flow rate: 13 ml/min, injection volume: 500 μl, temperature: 45° C.).

Enantiomer A: Rₜ=15.09 min; [α]_{D}^{20.0} (c=0.50; CHCl₃)=−18.9 Enantiomer B: Rₜ=17.46 min; [α]_{D}^{20.2} (c=0.51; CHCl₃)=+18.7 (Column: Daicel Chiralpak AS 10 μm 250×4.6 mm, eluent: 50% isohexane/50% isopropanol, flow rate: 0.5 m/min, injection volume: 5 μl, temperature: 42° C.).

In a similar manner, the following were prepared:

### Example 2

### 3,4-trans-3-[2-(2-Carbamoylmethoxy)phenyl]-2-[2-(1-cyclohexen-1-yl)ethyl]-1-oxo-N-(2-pyridinylmethyl)-1,2,3,4-tetrahydro-4-isoquinolinecarboxamide

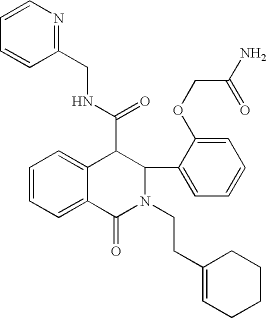

MS (ESI): 539 (M+H)⁺ ¹H NMR (300 MHz, DMSO): δ=1.42-1.51 (m, 4H); 1.82-1.86 (m, 4H); 2.01-2.06 (m, 2H); 2.68-2.78 (m, 1H); 3.92-4.02 (m, 1H); 4.19 (s, 1H); 4.39 (d, 2H); 4.53-4.66 (AB system, 2H); 5.29 (s, 1H); 5.62 (s, 1H); 6.60 (d, 1H); 6.79 (t, 1H); 6.99 (d, 1H); 7.15-7.25 (m, 4H); 7.39-7.44 (m, 2H); 7.60 (s, 1H); 7.67-7.73 (dt, 1H); 7.81 (s, 1H); 7.98-8.01 (m, 1H); 8.14 (t, 1H); 8.43 (d, 1H).

### Example 3

### 3,4-trans-N-Benzyl-3-[2-(carbamoylmethoxy)phenyl]-2-[2-(1-cyclohexen-1-yl)ethyl]-1-oxo-1,2,3,4-tetrahydro-4-isoquinolinecarboxamide

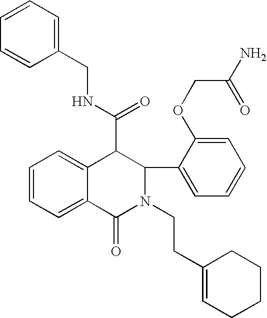

MS (ESI): 538 (M+H)⁺ ¹H NMR (300 MHz, DMSO): δ=1.44-1.62 (m, 4H); 1.82-1.88 (m, 4H); 1.98-2.03 (m, 2H); 2.66-2.76 (m, 1H); 3.89-3.98 (m, 1H); 4.13 (s, 1H); 4.29 (d, 2H); 4.52-4.65 (AB system, 2H); 5.29 (s, 1H); 5.56 (s, 1H); 6.59 (d, 1H); 6.75-6.80 (t, 1H); 6.98 (d, 1H); 7.18-7.29 (m, 7H); 7.39-7.41 (m, 2H); 7.62 (s, 1H); 7.83 (s, 1H); 7.95-7.99 (m, 1H); 8.13-8.18 (t, 1H).

### Example 4

### 3,4-trans-2-n-Butyl-3-[2-(carbamoylmethoxy)phenyl]-1-oxo-N-(3-pyridinylmethyl)-1,2,3,4-tetrahydro-4-isoquinolinecarboxamide

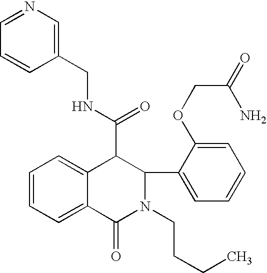

¹H NMR (300 MHz, CDCl₃): δ=0.91 (t, 3H); 1.24-1.34 (quin., 2H); 1.45-1.53 (m, 2H); 2.58-2.67 (quin., 1H); 4.04 (s, 1H); 4.09-4.23 (m, 1H); 4.27-4.34 (dd, 1H); 4.42-4.50 (dd, 1H); 4.52-4.69 (AB system, 2H); 5.62 (t, 1H); 5.73 (s, 1H); 5.82 (s, 1H); 6.75-6.87 (m, 3H); 7.07-7.10 (m, 1H); 7.21-7.23 (m, 2H); 7.43 (m, 3H); 8.25-8.28 (m, 1H); 8.41 (d, 1H); 8.50 (dd, 1H); 8.55 (s, 1H).

### Example 5

### 3,4-trans-3-[2-(Carbamoylmethoxy)phenyl]-1-oxo-2-(2-phenylethyl)-N-(3-pyridinylmethyl)-1,2,3,4-tetrahydro-4-isoquinolinecarboxamide

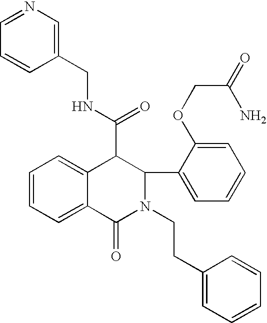

MS (ESI): 535 (M+H)⁺ ¹H NMR (200 MHz, CDCl₃): δ=2.71-2.99 (m, 3H); 4.06 (s, 1H); 4.21-4.46 (m, 3H); 4.50-4.71 (AB system, 2H); 5.63 (t, 1H); 5.75 (s, 1H); 5.91 (s, 1H); 6.77-6.87 (m, 3H); 7.07-7.24 (m, 8H); 7.41-7.47 (m, 3H); 8.25-8.30 (m, 1H); 8.40 (d, 1H); 8.46 (dd, 1.5 Hz; 1H); 8.53 (s, 1H).

### Example 6

### 3,4-trans-3-[2-(Carbamoylmethoxy)phenyl]-2-[2-(2-methoxyphenyl)ethyl]-1-oxo-N-(3-pyridinylmethyl)-1,2,3,4-tetrahydro-4-isoquinolinecarboxamide

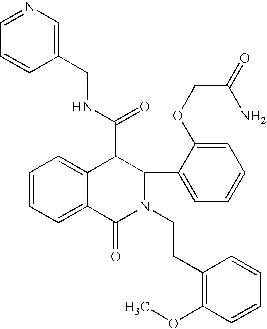

MS (ESI): 565 (M+H)⁺ ¹H NMR (200 MHz, CDCl₃): δ=2.81-2.87 (m, 3H); 3.79 (s, 3H); 4.05 (s, 1H); 4.24-4.49 (m, 3H); 4.51-4.73 (AB system, J_{AB}=14.5HZ, 2H); 5.61 (t, J=6 Hz, 1H); 5.76 (s, 1H); 5.99 (s, 1H); 6.77-6.88 (m, 5H); 7.07-7.21 (m, 5H); 7.44-7.50 (m, 3H); 8.26-8.30 (m, 1H); 8.40 (d, J=2 Hz, 1H); 8.47 (dd, J=5 Hz, 1.5 Hz, 1H); 8.68 (s, 1H).

### Example 7

### 3,4-trans-3-[2-(Carbamoylmethoxy)phenyl]-2-n-hexyl-1-oxo-N-(3-pyridinylmethyl)-1,2,3,4-tetrahydro-4-isoquinolinecarboxamide

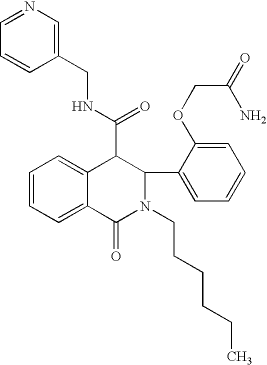

MS (ESI): 515 (M+H)⁺ ¹H NMR (200 MHz, CDCl₃): δ=0.84-0.90 (t, J=6.5 Hz, 3H); 1.27 (m, 6H); 1.49-1.52 (m, 2H); 2.56-2.70 (m, 1H); 4.04 (s, 1H); 4.09-4.53 (m, 3H); 4.50-4.70 (AB system, J_{AB}=14.5 Hz, 2H); 5.60 (t, J=6 Hz, 1H); 5.73 (s, 1H); 5.82 (s, 1H); 6.74-6.87 (m, 3H); 7.06-7.10 (m, 1H); 7.20-7.22 (m, 2H); 7.42-7.50 (m, 3H); 8.24-8.29 (m, 1H); 8.40 (d, J=1.5 Hz, 1H); 8.50 (dd, J=5 Hz, 1.5 Hz, 1H); 8.55 (s, 1H).

### Example 8

### 3,4-trans-3-[2-(Carbamoylmethoxy)phenyl]-2-[2-(3,4-dimethoxyphenyl)ethyl]-1-oxo-N-(3-pyridinylmethyl)-1,2,3,4-tetrahydro-4-isoquinolinecarboxamide

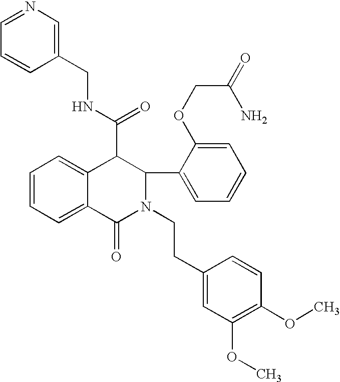

MS (ESI): 595 (M+H)⁺ ¹H NMR (200 MHz, CDCl₃): δ=2.68-3.00 (m, 3H); 3.83 (s, 3H); 3.87 (s, 3H); 4.06 (s, 1H); 4.23-4.48 (m, 3H); 4.50-4.71 (AB system, J_{AB}=14.5 Hz, 2H); 5.63 (t, J=6 Hz, 1H); 5.75 (s, 1H); 5.90 (s, 1H); 6.75-6.87 (m, 6H); 7.08-7.12 (m, 1H); 7.17-7.21 (m, 2H); 7.41-7.49 (m, 3H); 8.26-8.30 (m, 1H); 8.41 (d, J=1.5 Hz, 1H); 8.47 (dd, J=5 Hz, 1.5 Hz, 1H); 8.53 (s, 1H).

### Example 9

### 3,4-trans-3-[2-(Carbamoylmethoxy)phenyl]-2-[2-(4-methylphenyl)ethyl]-1-oxo-N-(3-pyridinylmethyl)-1,2,3,4-tetrahydro-4-isoquinolinecarboxamide

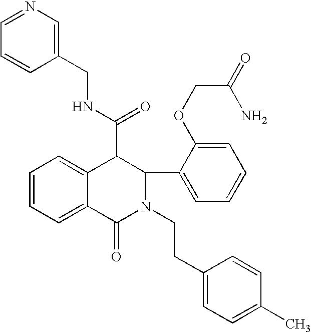

MS (ESI): 549 (M+H)⁺ ¹H NMR (200 MHz, CDCl₃): δ=2.29 (s, 3H); 2.66-2.96 (m, 3H); 4.05 (s, 1H); 4.22-4.46 (m, 3H); 4.50-4.71 (AB system, J_{AB}=14.5 Hz, 2H); 5.62 (t, J=6 Hz, 1H); 5.75 (s, 1H); 5.90 (s, 1H); 6.74-6.87 (m, 3H); 7.04-7.10 (m, 5H); 7.14-7.21 (m, 2H); 7.41-7.48 (m, 3H); 8.25-8.29 (m, 1H); 8.40 (d, J=2 Hz, 1H); 8.46 (dd, J=5 Hz, 1.5 Hz, 1H); 8.54 (s, 1 H).

### Example 10

### 3,4-trans-3-[2-(Carbamoylmethoxy)phenyl]-1-oxo-N-(3-pyridinylmethyl)-1,2,3,4-tetrahydro-2-(3,3,3-trifluoropropyl)-4-isoquinolinecarboxamide

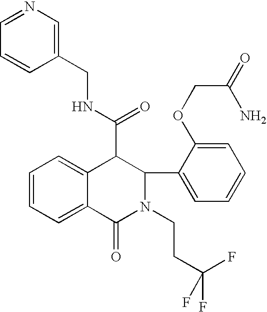

MS (ESI): 527 (M+H)⁺ ¹H NMR (200 MHz, CDCl₃): δ=2.26-2.46 (m, 2H); 2.82-2.96 (quin., J=7 Hz, 1H); 4.04 (s, 1H); 4.21-4.54 (m, 3H); 4.51-4.70 (AB system, J_{AB}=14.5 Hz, 2H); 5.66 (t, J=6 Hz, 1H); 5.74 (s, 1H); 5.84 (s, 1H); 6.70-6.89 (m, 3H); 7.09-7.13 (m, 1H); 7.21 (m, 2H); 7.43-7.52 (m, 3H); 8.24-8.28 (m, 1H); 8.40 (d, J=2 Hz, 2H); 8.51 (m, 1H).

### Example 11

### 3,4-trans-2-[2-( l -Cyclohexen-1-yl)ethyl]-1-oxo-3-{2-[2-oxo-2-(1-piperidinyl)ethoxy]phenyl}-N-(2-pyridinylmethyl)-1,2,3,4-tetrahydro-4-isoquinolinecarboxamide

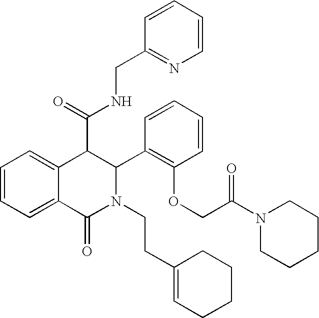

MS (ESI): 607 (M+H)⁺ ¹H NMR (300 MHz, DMSO): δ=1.43-1.57 (m, 10H); 1.77-1.91 (m, 4H); 1.96-2.09 (m, 2H); 2.57-2.75 (m, 1H); 3.40-3.51 (m, 4H); 3.90-3.99 (m, 1H); 4.28 (s, 1H); 4.28-4.51 (m, 2H); 4.94-5.05 (AB system, J_{AB}=14.5 Hz, 2H); 5.30 (s, 1H); 5.47 (s, 1H); 6.59 (d, J=7.5 Hz, 1H); 6.75-6.80 (m, 1H); 7.11-7.39 (m, 7H); 7.68-7.74 (dt, J=7.5 Hz, 1.5 Hz, 1H); 7.93-7.96 (m, 1H); 8.47 (d, J=4 Hz, 1H); 9.02-9.06 (t, J=6 Hz, 1H).

### Example 12

### 3,4-trans-2-[2-(1-Cyclohexen-1-yl)ethyl]-1-oxo-3-{2-[2-oxo-2-(1-piperidinyl)ethoxy]phenyl}-N-(3-pyridinylmethyl)-1,2,3,4-tetrahydro-4-isoquinolinecarboxamide

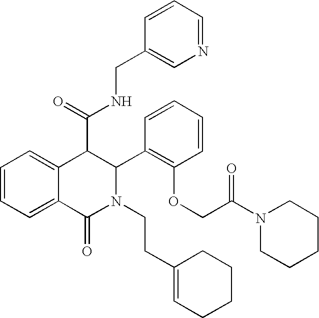

MS (ESI): 607 (M+H)⁺ ¹H NMR (300 MHz, DMSO): δ=1.49-1.59 (m, 10H); 1.78-1.88 (m, 6H); 2.58-2.63 (m, 1H); 3.46 (m, 4H); 3.86-4.02 (m, 1H); 4.20 (s, 1H); 4.37 (m, 2H); 4.90-5.06 (m, 2H); 5.26 (s, br, 1H); 5.33 (s, 1H); 6.56 (d, J=7.5 Hz, 1H); 6.73-6.79 (m, 1H); 7.05-7.09 (m, 1H); 7.14-7.19 (m, 2H); 7.28-7.38 (m, 3H); 7.69-7.73 (m, 1H); 7.91-7.96 (m, 1H); 8.43 (dd, J=4.5 Hz, 1.5 Hz, 1H); 8.54 (d, J=1.5 Hz, 1H); 9.19-9.25 (t, J=6 Hz, 1H).

### Example 13

### 3,4-trans-2-[2-(1-Cyclohexen-1-yl)ethyl]-3-{2-[2-(4-methyl-1-piperazinyl)-2-oxoethoxy]phenyl}-1-oxo-N-(3-pyridinylmethyl)-1,2,3,4-tetrahydro-4-isoquinolinecarboxamide

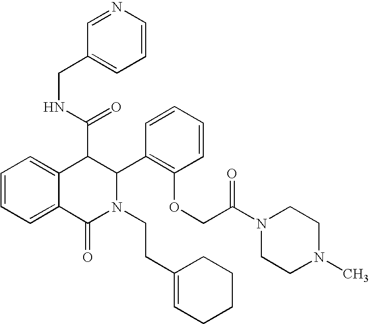

MS (ESI): 622 (M+H)⁺ ¹H NMR (300 MHz, DMSO): δ=1.43-1.51 (m, 4H); 1.78-1.94 (m, 6H); 2.21 (s, 3H); 2.31-2.37 (m, 4H); 2.53-2.64 (m, 1H); 3.50 (m, 4H); 3.88-3.97 (m, 1H); 4.19 (s, 1H); 4.30-4.44 (m, 2H); 4.94-5.05 (AB system, J_{AB}=15 Hz, 2H); 5.26 (s, 1H); 5.33 (s, 1H); 6.56 (d, J=7.5 Hz, 1H); 6.74-6.79 (m, 1H); 7.05-7.09 (m, 1H); 7.13-7.22 (m, 2H); 7.29-7.37 (m, 3H); 7.69 (m, 1H); 7.94 (m, 1H); 8.43 (dd, J=4.5 Hz, 1.5 Hz, 1H); 8.53 (d, J=1.5 Hz, 1H); 9.08-9.12 (t, J=6 Hz, 1H).

### Example 14

### 3,4-trans-2-[2-(1-Cyclohexen-1-yl)ethyl]-3-{2-[2-(isopropylamino)-2-oxoethoxy]phenyl}-1-oxo-N-(2-pyridinylmethyl)-1,2,3,4-tetrahydro-4-isoquinolinecarboxamide

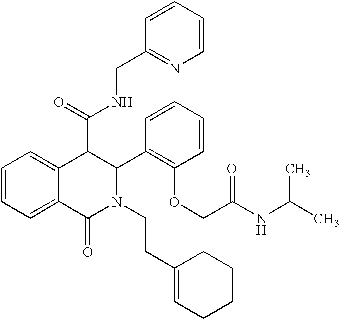

MS (ESI): 581 (M+H)⁺ ¹H NMR (200 MHz, DMSO): δ=1.01 (d, J=6.5 Hz, 3H); 1.12 (d, J=6.5 Hz, 3H); 1.41-1.57 (m, 4H); 1.77-1.91 (m, 4H); 2.01-2.09 (m, 2H); 2.58-2.72 (m, 1H); 3.17 (d, J=5 Hz, 1H); 3.93-4.11 (m, 2H); 4.15 (s, 1H); 4.28-4.47 (m, 2H); 4.50-4.70 (AB system, J_{AB}=14 Hz, 2H); 5.30 (s, b, 1H); 5.67 (s, 1H); 6.60 (m, 1H); 6.76-6.83 (t, J=7.5 Hz, 1H); 6.99 (d, J=8 Hz, 1H); 7.15-7.29 (m, 3H); 7.43-7.47 (t, J=4 Hz, 2H); 7.66-7.74 (dt, J=7.5 Hz, 1.5 Hz, 1H); 7.98-8.18 (m, 3H); 8.43 (d, J=5 Hz, 1H).

### Example 15

### 3,4-trans-2-[2-(1-Cyclohexen-1-yl)ethyl]-1-oxo-3-{2-[2-oxo-2-(1-piperidinyl)ethoxy]phenyl}-N-(4-pyridinylmethyl)-1,2,3,4-tetrahydro-4-isoquinolinecarboxamide

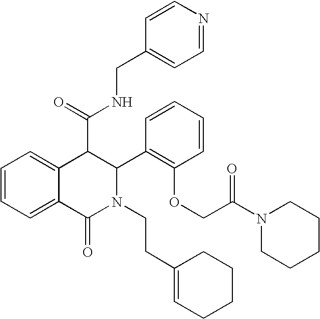

MS (ESIpos): m/z=607 (M+H)⁺ ¹H NMR (300 MHz, DMSO-d₆): δ=1.39-1.65 (m, 10H), 1.74-2.03 (m, 6H), 2.58-2.74 (m, 1H), 3.45 (d, 4H), 3.89-4.00 (m, 1H), 4.24 (s, 1H), 4.30-4.45 (m, 2H), 4.93-5.06 (m, 2H), 5.27 (s, 1H), 5.41 (s, 1H), 6.57 (d, 1H), 6.77 (dt, 1H), 7.09-7.22 (m, 3H), 7.29 (d, 2H), 7.34-7.39 (m, 2H), 7.91-7.97 (m, 1H), 8.47 (d, 2H), 9.17 (t, 1H).

### Example 16

### 3,4-trans-2-[2-(1-Cyclohexen-1-yl)ethyl]-3-{2-[2-(isopropylamino)-2-oxoethoxy]phenyl}-1-oxo-N-(3-pyridinylmethyl)-1,2,3,4-tetrahydro-4-isoquinolinecarboxamide

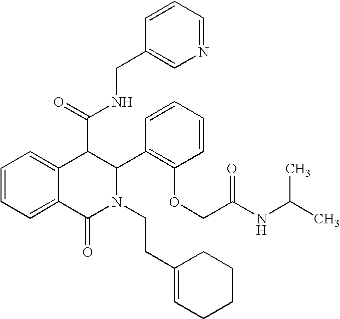

MS (ESIpos): m/z=581 (M+H)⁺ ¹H NMR (300 MHz, DMSO-d₆): δ=1.07 (d, 3H), 1.16 (d, 3H), 1.40-1.55 (m, 4H), 1.78-2.0 (m, 6H), 2.52-2.64 (m, 1H), 3.94-4.11 (m, 3H), 4.23-4.39 (m, 2H), 4.63 (quart., 1H), 5.28 (s, 1H), 5.56 (s, 1H), 5.76 (s, 1H), 6.57 (d, 1H), 6.78 (t, 1H), 6.98 (d, 1H), 7.17-7.23 (m, 2H), 7.27-7.31 (m, 1H), 7.40-7.45 (m, 2H), 7.58-7.61 (m, 1H), 7.97-8.00 (m, 1H), 8.19-8.25 (m, 2H), 8.40-8.46 (m, 2H).

HPLC Method:

Column: Kromasil C18 60*2;

injection volume 1.00 μl;

flow rate: 0.75 ml/min;

eluent: A=5 ml of HClO₄/l H₂O,

B=CH₃CN;

gradient [t (min): A/B]: 0.5: 98/2; 4.5: 10/90; 6.5: 10/90; 6.7: 98/2; 7.5: 98/2.

LC-MS Method A:

Column: Symmetry C18;

injection volume: 5 μl;

flow rate: 0.5 ml/min;

eluent: A=CH₃CN+0.1 % of formic acid,

B=H₂O+0.1 % of formic acid;

gradient [t (min): A/B]: 0.0: 10/90; 4.0: 90/10; 6.0: 90/10; 6.1: 10/90 (flow rate 1.0 ml/min); 7.5: 10/90.

MS: ESI.

LC-MS method B:

Column: Symmetry C18;

injection volume: 2 μl;

flow rate: 0.9 ml/min;

eluent: A=CH₃CN,

B=0.3 g of 30% HCl/l of H₂O;

gradient [t (min): A/B]: 0.0: 10/90; 3.0: 90/10 (flow rate: 1.2 ml/min); 6.0: 90/10 (flow rate: 1.2 ml/min).

MS: ESI.

1. Compounds of the general formula (I) 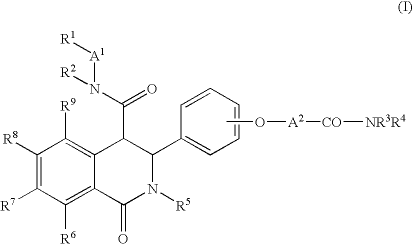
in which
R¹ is phenyl or 5- or 6-membered heteroaryl, where phenyl and heteroaryl may optionally be identically or differently substituted by radicals selected from the group of halogen, formyl, carbamoyl, cyano, hydroxyl, trifluoromethyl, trifluoromethoxy, nitro, (C₁-C₆)alkyl, (C₁-C₆)alkoxy and (C₁-C₆)alkylthio,
A¹ is a bond or (C₁-C₆)alkanediyl,
R² is hydrogen or (C₁-C₆)alkyl,
R³ and R⁴ are the same or different and are each hydrogen, (C₁-C₈)alkyl or (C₃-C₈)cycloalkyl,
or
R³ and R⁴ together with the adjacent nitrogen atom are a radical selected from the group of pyrrolidyl, piperidyl, azepinyl and piperazinyl, where the radicals may optionally be substituted by methyl or ethyl,
A² is (C₁-C₆)alkanediyl,
R⁵ is (C₁-C₈)alkyl which may optionally be substituted by a radical selected from the group of trifluoromethyl, halogen, saturated or partly unsaturated (C₃-C₈)cycloalkyl and (C₆-C₁₀)aryl, where aryl may itself optionally be substituted identically or differently by radicals selected from the group of halogen, formyl, carbamoyl, cyano, hydroxyl, trifluoromethyl, trifluoromethoxy, nitro, (C₁-C₆)alkyl, (C₁-C₆)alkoxy and (C₁-C₆)alkylthio,
and
R⁶, R⁷, R⁸ and R⁹ are the same or different and are each hydrogen, halogen, formyl, carbamoyl, cyano, hydroxyl, trifluoromethyl, trifluoromethoxy, nitro, (C₁-C₆)alkyl, (C₁-C₆)alkoxy or (C₁-C₆)alkylthio, and their salts, hydrates and/or solvates.

2. Compounds as claimed in claim 1, wherein
R¹ is 3-pyridyl which is optionally identically or differently substituted by chlorine, fluorine or methyl,
A¹ is methylene,
R² is hydrogen,
R³ and R⁴ are each hydrogen,
A² is methylene,
R⁵ is (C₁-C₃)alkyl which is substituted by a radical selected from the group of trifluoromethyl, unsaturated or monounsaturated (C₅-C₇)-cycloalkyl and optionally methyl-, halogen- or methoxy-substituted phenyl, or
is (C₄-C₆)alkyl which is optionally substituted by trifluoromethyl, and
R⁶, R⁷, R⁸ and R⁹ are each hydrogen,
and their salts, hydrates and/or solvates.

3. A process for preparing compounds of the general formula (I) as claimed in claim 1, characterized in that compounds of the general formula (VI) 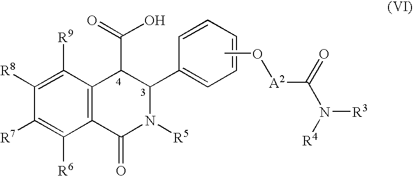
in which
A², R³, R⁴, R⁵, R⁶, R⁷, R⁸ and R⁹ are each as defined in claim 1 are reacted with compounds of the general formula (VII)
R¹—A¹—NH—R² (VII)
in which
A¹, R¹ and R² are each as defined in claim 1.

4. Compounds as claimed in claim 1 or 2 for the treatment and/or prophylaxis of diseases.

5. A pharmaceutical comprising at least one of the compounds as claimed in claim 1 or 2 in admixture with at least one pharmaceutically acceptable, substantially nontoxic carrier or excipient.

6. The use of compounds as claimed in claim 1 or 2 for producing a pharmaceutical for the treatment and/or prophylaxis of states of pain.

7. The pharmaceutical as claimed in claim 5 for the treatment and/or prophylaxis of states of pain.
